(19) United States
(12) Patent Application Publication
(10) Pub. No.: US 2024/0095914 A1
(43) Pub. Date: Mar. 21, 2024

(54) INTERPRETATION MANAGEMENT APPARATUS, INTERPRETATION MANAGEMENT SYSTEM, IMAGE MANAGEMENT APPARATUS, INTERPRETATION TERMINAL, ANALYSIS APPARATUS, INTERPRETATION MANAGEMENT METHOD, AND RECORDING MEDIUM

(71) Applicant: Konica Minolta, Inc., Tokyo (JP)

(72) Inventors: Shinsuke KATSUHARA, Tokyo (JP); Yozo OKUBO, Tokyo (JP)

(21) Appl. No.: 18/466,358

(22) Filed: Sep. 13, 2023

(30) Foreign Application Priority Data
Sep. 15, 2022 (JP) 2022-147093

(51) Int. Cl. G06T 7/00; G16H 40/20

(52) U.S. Cl. CPC G06T 7/0012; G16H 40/20; G06T 2207/30096

(57) ABSTRACT

An interpretation management apparatus including a hardware processor that: acquires an automatically generated finding obtained by computer processing on medical information; acquires a first interpretation finding created by a user based on the medical information; determines whether or not a result combination is a combination for which an active notification is to be performed, the result combination being a combination of a result of the automatically generated finding and a result of the first interpretation finding; and actively notifies predetermined information in response to determination that the result combination is the combination for which the active notification is to be performed.

### CROSS-REFERENCE TO RELATED APPLICATIONS

The entire disclosure of Japanese Patent Application No. 2022-147093, filed on Sep. 15, 2022, including description, claims, drawings and abstract is incorporated herein by reference.

### BACKGROUND

### Technical Field

The present invention relates to an interpretation management apparatus, an interpretation management system, an image management apparatus, an interpretation terminal, an analysis apparatus, an interpretation management method, and a recording medium.

### Description of Related Art

In recent years, with the development of artificial intelligence (AI) technology, analysis by AI has been introduced also in the medical field, and attempts have been made to support analysis and diagnosis of medical information such as image diagnosis, which have been conventionally performed by doctors, by using AI.

At a clinical site of medical care, it is required to reduce a burden on a doctor by appropriately and quickly performing examination and diagnosis and making the diagnosis efficient and optimal. The introduction of AI analysis is expected to contribute to the efficiency and optimization of such diagnosis.

JP 2021-178110 A discloses an apparatus that performs priority notification (triage notification) for notifying a user in advance of interpretation based on an analysis result of AI for a medical image. The analysis of AI includes analysis of computer aided diagnosis (CAD).

In the case of such a triage type interpretation style, a doctor receives priority notification of all of them that are determined to be positive by AI. That is, as a way of thinking of triage, those that are determined to be positive by AI are naturally shown to a doctor as having a high priority. A case where AI determines positive is a case where AI has detected an abnormality and has a high possibility of a disease such as cancer.

On the other hand, JP 2017-010577 A describes that, in a Lung Cancer Examination between an AI and a doctor, when a primary interpretation by a primary interpretation doctor is finished, a secondary interpretation doctor evaluates CAD findings and the results of interpretation findings by the primary interpretation doctor. As described in JP 2017-010577 A, the interpretation style of taking some action by comparing the analysis result of the AI with the interpretation result of the doctor is referred to as a quality assurance (QA) type interpretation style.

As described above, as the concept of the triage type interpretation style, those determined as positive by AI is shown to the doctor as having a high priority. Therefore, in a case where a triage type interpretation style such as JP 2021-178110 A is combined with a QA type interpretation style such as JP 2017-010577 A, naturally, it is conceivable to increase the efficiency of interpretation by providing a triage notification only for those determined to be positive by AI and which also determined to be positive by the primary interpretation doctor.

However, as a result of examination by the present inventors, it has been found that there is a need for checking, with priority, an interpretation result determined to be positive by AI and determined to be negative by a primary interpretation doctor from the viewpoint of preventing overlooking of interpretation by the primary interpretation doctor, depending on the secondary interpretation doctor in the QA type interpretation style. In addition, the present inventors have found that there is a need for a secondary interpretation doctor to confirm, with priority, a case where interpretation results of a primary interpretation doctor and an AI are different in a QA-type interpretation style. That is, the inventors have found a need that, in the QA type interpretation style, a desired combination of active notifications among combinations of a result of findings automatically generated by computer processing (an analysis result of AI) and a result of interpretation findings differs depending on medical facilities or doctors. Here, the active notification is a notification that is received and displayed even if a user (doctor) does not try to display information by himself/herself.

### SUMMARY

The present invention is directed to enabling active notification in a case where a combination of a result of automatically generated finding obtained by computer processing and a result of interpretation finding by a doctor is a combination to be actively notified.

To achieve at least one of the abovementioned objects, according to an aspect of the present invention, a interpretation management apparatus reflecting one aspect of the present invention is an interpretation management apparatus including a hardware processor that: acquires an automatically generated finding obtained by computer processing on medical information; acquires a first interpretation finding created by a user based on the medical information; determines whether or not a result combination is a combination for which an active notification is to be performed, the result combination being a combination of a result of the automatically generated finding and a result of the first interpretation finding; and actively notifies predetermined information in response to determination that the result combination is the combination for which the active notification is to be performed.

### BRIEF DESCRIPTION OF THE DRAWINGS

The advantages and features provided by one or more embodiments of the invention will become more fully understood from the detailed description given hereinafter and the appended drawings which are given by way of illustration only, and thus are not intended as a definition of the limits of the present invention, and wherein:

FIG. 1 is an overall configuration diagram of an interpretation management system;

FIG. 2 is a block diagram showing a functional configuration of an analysis apparatus;

FIG. 3 is a block diagram showing a functional configuration of the image management apparatus;

FIG. 4 is a block diagram showing a functional configuration of an interpretation terminal;

FIG. 5 is a flowchart showing a flow of a notification setting process executed by the controller of FIG. 3;

FIG. 6 is a diagram showing an example of a notification setting screen;

FIG. 7 is a flowchart showing the flow of notification control process executed by the controller of FIG. 3;

FIG. 8 illustrates an example of active notification;

FIG. 9 illustrates an example of active notification;

FIG. 10 is a diagram illustrating an example of an examination list screen on which a passive notification is displayed; and

FIG. 11 is a diagram showing a modification of the notification setting screen.

### DETAILED DESCRIPTION

Hereinafter, preferred embodiments according to the present invention will be described with reference to the drawings. The present invention is not limited to the example shown in the drawing.

[Configuration of Interpretation Management System]

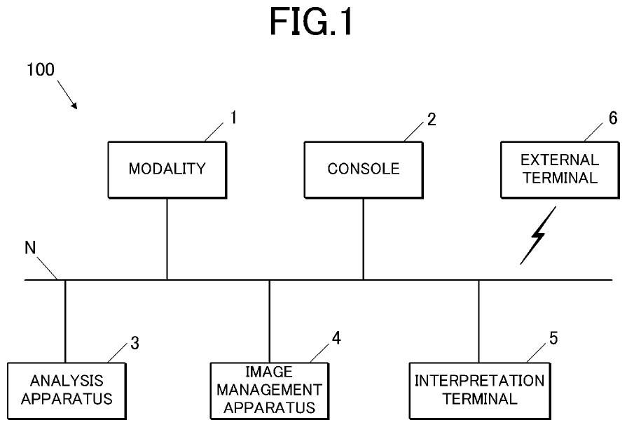

FIG. 1 illustrates a system configuration example of an interpretation management system 100 according to the first embodiment.

As illustrated in FIG. 1, the interpretation management system 100 includes a modality 1, a console 2, an analysis apparatus 3, an image management apparatus 4, an interpretation terminal 5, an external terminal 6, and the like. The apparatuses included in the interpretation management system 100 are connected to each other via a communication network such as a local area network (LAN), a wide area network (WAN), or the Internet. Among the devices constituting the interpretation management system 100, the modality 1, the console 2, the analysis apparatus 3, the image management apparatus 4, and the interpretation terminal 5 are connected to an in-hospital network N. These devices conform to the HL7 (Health Level Seven) or DICOM (Digital Image and Communications in Medicine) standard. Communication between these devices is performed according to HL7 or DICOM. The external terminal 6 may be directly connected to the hospital network N, or may be connected from an external network via a router (not illustrated). The number of modalities 1, consoles 2, analysis apparatuses 3, interpretation terminals 5, external terminals 6, and the like is not particularly limited.

(Configuration of Modality And Console)

The modality 1 is an image generation apparatus such as an X-ray imaging apparatus (DR, CR), an ultrasound diagnostic apparatus (US), CT, or MRI, for example. Based on examination order information transmitted from a radiology information system (RIS) or the like (not illustrated), the modality 1 takes an image of an examination target site of a patient as a subject to generate a medical image as medical information, and outputs the medical image to the console 2.

The console 2 includes a controller, a display part, an operation part, a communication unit, a storage section, and the like, which are not illustrated. The console 2 is an imaging control device that controls imaging in the modality 1. The console 2 outputs imaging conditions and image reading conditions to the modality 1 on the basis of examination order information transmitted from an RIS or the like (not illustrated), and acquires image data of a medical image captured by the modality 1. Furthermore, the console 2 writes accompanying information to each of the medical images acquired from the modality 1 in accordance with the DICOM standard, and transmits the medical images with the accompanying information to the analysis apparatus 3 and the image management apparatus 4. For example, the console 2 writes the accompanying information to a header of an image file of the medical image. The accompanying information includes, for example, patient information, examination information, and image identification information. The patient information includes a patient ID, patient name, date of birth, age, sex, height, weight, and the like. The examination information includes an examination ID, an examination date and time, a type of modality, an examination site, an examination type, a request department, and an examination purpose. The image identification information is a unique ID (UID) or the like.

(Configuration of Analysis Apparatus)

The analysis apparatus 3 performs analysis (lesion detection) by computer processing on the medical image transmitted from the console 2, generates an automatically generated finding as an analysis result (detection result), and transmits (outputs) the automatically generated finding to the image management apparatus 4.

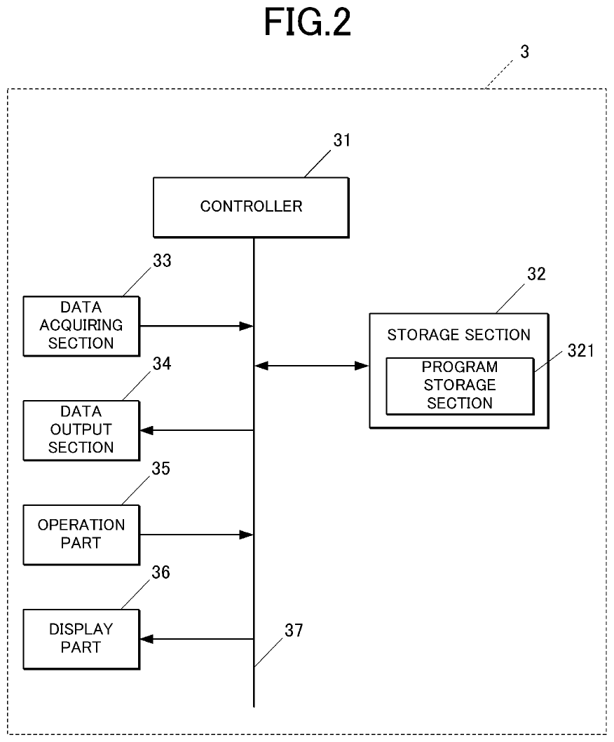

FIG. 2 is a block diagram illustrating the functional configuration of the analysis apparatus 3.

As illustrated in FIG. 2, the analysis apparatus 3 comprises a controller (hardware processor) 31, a storage section 32, a data acquiring section 33, a data output section 34, an operation part 35, a display part 36, and the like. The respective units of the analysis apparatus 3 are connected by a bus 37.

The controller 31 includes a CPU (Central Processing Unit), a RAM (Random Access Memory), and the like.

The controller 31 controls the overall operation of each unit of the analysis apparatus 3. Specifically, the CPU of the controller 31 reads various processing programs stored in a program storage section 321 of the storage section 32, loads the programs to a RAM, and executes various types of processing in accordance with the programs.

For example, the controller 31 performs analysis (lesion detection) by computer processing on the medical image acquired by the data acquiring section 33 and generates an automatically generated finding. Then, the controller 31 adds the image identification information of the medical image of the analysis source to the generated automatically generated finding, and transmits (outputs) them to the image management apparatus 4 by the data output section 34. Here, as the computer processing, for example, AI analysis using artificial intelligence (AI) that performs image diagnosis and image analysis including detection of a lesion by computer aided diagnosis (CAD) and detection of a lesion by machine learning is used.

In the present embodiment, the automatically generated finding includes information of “+” indicating that there is an abnormal finding (lesion) or information of “−” indicating that there is no abnormal finding. If there is an abnormal finding, information about the detected position of a lesion, the type and number of abnormal findings, and the like are also included. Furthermore, information on the certainty degree of the result of the automatically generated finding is also included.

The storage section 32 is configured by a hard disk drive (HDD), a semiconductor memory, or the like. The storage section 32 includes a program storage section 321 that stores programs for executing various kinds of processing including an analysis program for analyzing medical information and generating an automatically generated finding. The storage section 32 also stores parameters, files, and the like required for execution of the programs stored in the program storage section 321.

The data acquiring section 33 is composed of, for example, a network interface. The data acquiring section 33 is configured to receive data from an external device connected via the in-hospital network N (and an external network) in a wired or wireless manner. In the present embodiment, the data acquiring section 33 is configured by a network interface or the like, but can also be configured by a port or the like into which a USB memory, an SD card, or the like can be inserted.

The data output section 34 includes, for example, a network interface. The data output section 34 is configured to output data to an external device connected via the in-hospital network N (and an external network) in a wired or wireless manner. Note that in the present embodiment, the data output section 34 is assumed to be formed with a network interface and the like, but a connector for connecting to an external device, ports for various media such as a USB memory and the like are applicable.

The operation part 35 includes a keyboard having various keys, a pointing device such as a mouse, or a touch screen attached to the display part 36. The operation part 35 outputs, to the controller 31, an operation signal input by a key operation on the keyboard, a mouse operation, or a touch operation on the touch screen.

The display part 36 includes a monitor such as a liquid crystal display (LCD). The display part 36 displays various screens according to an instruction of a display signal input from the controller 31. Note that the number of monitors is not limited to one, and a plurality of monitors may be provided.

The analysis apparatus 3 may be configured integrally with the image management apparatus 4.

(Configuration of Image Management Apparatus)

The image management apparatus 4 is, for example, a server of a picture archiving and communication system (PACS). The image management apparatus 4 stores and manages the image data of the medical image output from the console 2, the automatically generated finding output from the analysis apparatus 3, and the interpretation finding output from the interpretation terminal 5. In the present embodiment, the image management apparatus 4 functions as an interpretation management apparatus.

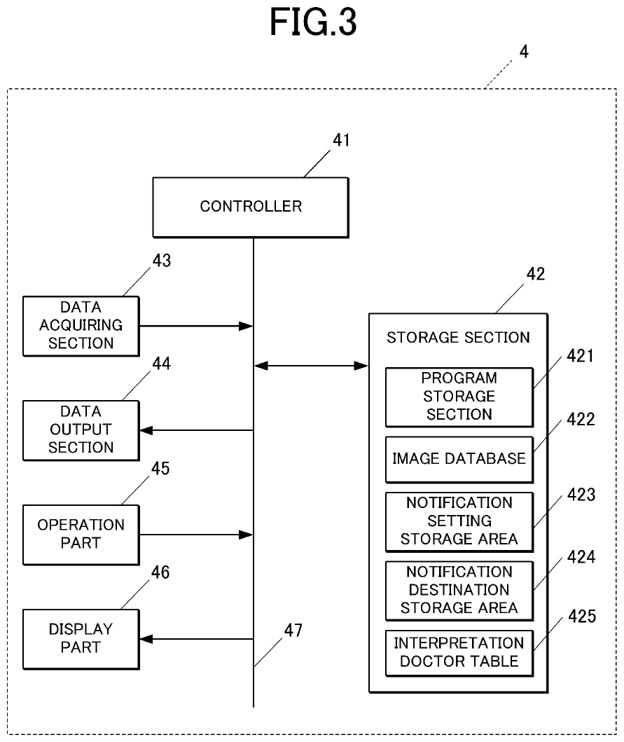

FIG. 3 is a block diagram illustrating a functional configuration of an image management apparatus 4.

As illustrated in FIG. 3, the image management apparatus 4 includes a controller (hardware processor) 41, a storage section 42, a data acquiring section 43, a data output section 44, an operation part 45, a display part 46, and the like. The respective units of the image management apparatus 4 are connected by a bus 47.

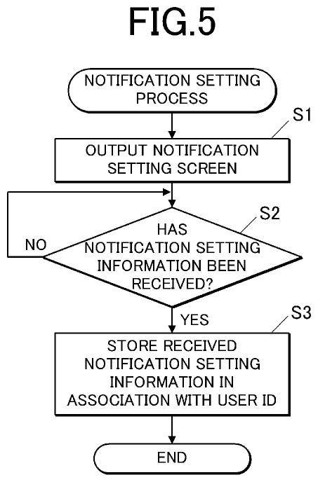

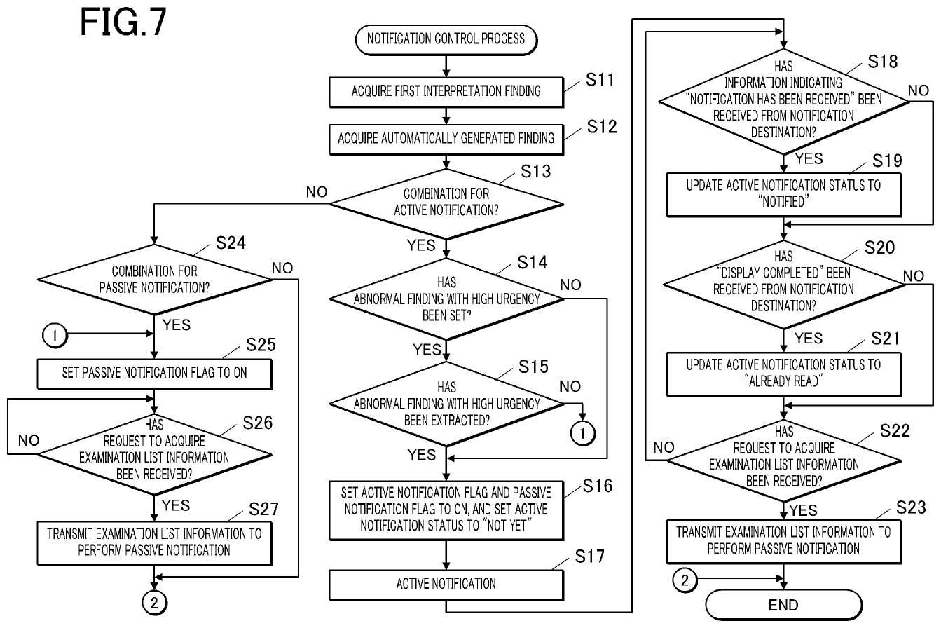

The controller 41 includes a central processing unit (CPU), a random access memory (RAM), and the like. The controller 41 integrally controls the operation of each unit of the image management apparatus 4. Specifically, the CPU of the controller 41 reads various processing programs stored in a program storage section 421 of the storage section 42, loads the programs to a RAM, and executes various types of processing in accordance with the programs. The storage section 42 is configured by a hard disk drive (HDD), a semiconductor memory, or the like. The storage section 42 includes a program storage section 421 that stores a program for executing various processes including a program for executing the notification setting process illustrated in FIG. 5 and the notification control process illustrated in FIG. 7. The storage section 42 also stores parameters, files, and the like required for execution of the program stored in the program storage section 421.

Furthermore, the storage section 42 includes an image database (DB) 422. The image database 422 is a database for storing a medical image, an automatically generated finding for the medical image, a first interpretation finding for the medical image, and a second interpretation finding for the medical image in association with each other. The first interpretation finding is an interpretation finding created by the user (primary interpretation doctor). The second interpretation finding is an interpretation finding created by the user (secondary interpretation doctor). The image database 422 has storage areas for the medical image, the automatically generated findings, the first interpretation findings, and the second interpretation findings. The image database 422 has an image management table for storing management information of the medical image stored in the image database 422. The image management table is provided with an area for storing information such as patient information of a medical image, examination information, image identification information, primary interpretation doctor information, secondary interpretation doctor information, status information of primary interpretation, status information of secondary interpretation, an active notification flag, a passive notification flag, status information of active notification, an AI determination result, a user determination result, identification information of an automatically generated finding, identification information of a first interpretation finding, and identification information of a second interpretation finding. The identification information of the automatically generated finding is, for example, a file name of the automatically generated finding. The identification information of the first interpretation finding is, for example, the file name of the first interpretation finding. The identification information of the second interpretation finding is, for example, the file name of the second interpretation finding.

For example, “not interpreted”, “during interpretation”, “interpreted”, and the like are stored in the area of the status information of the primary interpretation according to the state of the primary interpretation.

In the area of the status information of the secondary interpretation, for example, “not interpreted”, “waiting for interpretation”, “during interpretation”, “interpreted (approved)”, or the like is stored according to the state of the secondary interpretation.

In the area of the active notification flag, a flag indicating whether or not the combination of the result of the automatically generated finding of the medical image and the result of the primary interpretation finding has been determined to be a combination for active notification by notification control process (see FIG. 7) to be described later is stored. For example, OFF is stored in a case where it is determined that the combination is not a combination for active notification, and ON is stored in a case where it is determined that the combination is a combination for active notification.

In the area of the passive notification flag, a flag indicating whether or not the combination of the result of the automatically generated finding of medical image and the result of the primary interpretation finding has been determined to be a combination to be passively notified by the notification control process (see FIG. 7) to be described later is stored. For example, OFF is stored when it is determined that the combination is not a combination to be passively notified, and ON is stored when it is determined that the combination is a combination to be passively notified.

In the area of the status information of the active notification, information indicating whether the active notification is not received (“not yet”), the active notification is received (“notified”), or the display of the active notification is completed (“already read”) in the terminal device (first external terminal) of the active notification destination is stored.

In the area of the AI determination result, the presence or absence of an abnormal finding due to an automatically generated finding is stored. If there is an abnormal finding due to an automatically generated finding, the number of abnormal findings is also stored in the AI determination result area.

The presence or absence of an abnormal finding due to the first interpretation finding is stored in the user determination result area. In a case where there is an abnormal finding due to the first interpretation finding, the number of abnormal findings is also stored in the user determination result area.

Here, the active notification refers to a notification that is received and displayed even if a doctor (a secondary interpretation doctor in the present embodiment) who is a user does not try to display information by himself/herself. For example, a pop-up/blinking notification on the interpretation terminal 5, a push notification to the external terminal 6, and the like are included.

The passive notification refers to a notification that is displayed when a doctor (a secondary interpretation doctor in the present embodiment) who is a user tries to display it by himself/herself, that is, a notification that is not displayed unless the doctor tries to display it. For example, the passive notification includes a notification that is not displayed unless a doctor performs an operation of activating a predetermined application or displaying a predetermined screen (e.g., an examination list screen).

A notification setting storage area 423 is provided in the storage section 42. In the notification setting storage area 423, the user ID of the user who has performed the setting (the user ID of the secondary interpretation doctor) and notification setting information set in notification setting process (see FIG. 5) to be described later are stored in association with each other.

The storage section 42 is provided with a notification destination storage area 424. The notification destination storage area 424 stores the user ID of a secondary interpretation doctor and information on a notification destination (first external terminal) of an active notification in association with each other. Examples of the notification destination of the active notification include the interpretation terminal 5 used by the secondary interpretation doctor and the external terminal 6 carried by the secondary interpretation doctor. Further, the console 2 may be the notification destination.

An interpretation doctor table 425 is stored in the storage section 42. The interpretation doctor table 425 is, for example, a table in which the type of modality is associated with information (user ID or the like) of a primary interpretation doctor and a secondary interpretation doctor who interpret a medical image captured by the modality.

The interpretation doctor table 425 may store the combination of the type of modality and the request department in association with information on the primary interpretation doctor and the secondary interpretation doctor.

The storage section 42 also stores user information on users who can access the interpretation management system 100. Examples of the user information include a user ID, a password, a user name, a department to which the user belongs, and a mail (email) address.

The data acquiring section 43 is composed of, for example, a network interface. The data acquiring section 43 is configured to receive data from an external device connected via the in-hospital network N (and an external network) in a wired or wireless manner. In the present embodiment, the data acquiring section 43 is configured by a network interface or the like, but can also be configured by a port or the like into which a USB memory, an SD card, or the like can be inserted.

The data output section 44 includes, for example, a network interface. The data output section 44 is configured to output data to an external device connected via the in-hospital network N (and an external network) in a wired or wireless manner. In the present embodiment, the data output section 44 is configured by a network interface or the like, but a connector for connecting to an external device, a port of various media such as a USB memory, or the like can be applied.

The operation part 45 includes a keyboard having various keys, a pointing device such as a mouse, or a touch screen attached to the display part 46. The operation part 45 outputs, to the controller 41, an operation signal input by a key operation on the keyboard, a mouse operation, or a touch operation on the touch screen.

The display part 46 includes a monitor such as a liquid crystal display (LCD). The display part 46 displays various screens according to an instruction of a display signal input from the controller 41. Note that the number of monitors is not limited to one, and a plurality of monitors may be provided.

For example, the controller 41 of the image management apparatus 4 causes the data acquiring section 43 to acquire (receive) a medical image transmitted from the console 2, and stores the acquired medical image in the image database 422. Further, the controller 41 stores patient information, examination information, and image identification information, which are accompanying information of the acquired medical image, in the image management table of the image database 422. Further, the controller 41 stores the status information (not interpreted) of the primary interpretation and the status information (not interpreted) of the secondary interpretation in the image management table. Further, the controller 41 refers to the interpretation doctor table 425 and decides the primary interpretation doctor and the secondary interpretation doctor who interpret the acquired medical image. Then, the controller 41 stores the primary interpretation doctor information and the secondary interpretation doctor information in the record related to the medical image in the image management table.

Furthermore, the controller 41 causes the data acquiring section 43 to acquire (receive) the automatically generated finding and the image identification information transmitted from the analysis apparatus 3, and stores the acquired automatically generated finding in the image database 422. Further, the controller 41 searches the image management table for a record having the acquired image identification information, and stores the identification information of the acquired automatically generated finding in the searched record.

When the controller 41 receives a request to acquire examination list information from one of the interpretation terminal 5 with data acquiring section 43, the controller 41 extracts a record satisfying a predetermined condition from the image management table of the image database 422 and generates examination list information. Then, the controller 41 transmits the generated examination list to the interpretation terminal 5 through the data output section 44. Examples of the record matching the predetermined condition include a record in which the examination date and time is within a predetermined period, a record in which the type of modality is a predetermined type, and a record in which the secondary interpretation status information is not “interpretation completed”.

Furthermore, when the controller 41 receives, from the interpretation terminal 5, a request for acquiring a medical image of an examination whose status information on the primary interpretation doctor is “not interpreted” with the data acquiring section 43, the controller 41 reads, from the image database 422, the medical image and the automatically generated finding of the requested examination. The controller 41 transmits the read medical image and automatically generated findings to the interpretation terminal 5 through the data output section 44. Then, the controller 41 updates the status information of the primary interpretation of the record of the examination (medical image) in the image management table to “during interpretation”.

Furthermore, when the controller 41 acquires (receives) the first interpretation result and the image identification information from the interpretation terminal 5 by using the data acquiring section 43, the controller 41 stores the received first interpretation result in the image database 422. The controller 41 searches the image management table for a record having the acquired image identification information and stores the acquired identification information of the first interpretation finding in the searched record. Further, the controller 41 updates the status information of the primary interpretation of the record corresponding to the acquired image identification information to “interpreted”, and the status information of the secondary interpretation to “waiting for interpretation”. Then, the controller 41 executes the notification control process shown in FIG. 7. In the notification control process, the controller 41 acquires the received first interpretation finding and also acquires the corresponding automatically generated finding from the image database 422. The controller 41 determines whether or not the combination of the result of the automatically generated finding and the result of the first interpretation finding is a combination for active notification. In a case where it is determined that the combination is the combination for active notification, the controller 41 actively notifies predetermined information.

When the data acquiring section 43 receives a notification setting request from the interpretation terminal 5, the controller 41 executes the notification setting process shown in FIG. 5. In the notification setting process, the controller 41 causes the data output section 44 to output screen information on the notification setting screen 561 to the interpretation terminal 5. The notification setting screen 561 is a screen for enabling a combination to be actively notified (and a combination to be passively notified) among combinations of the result of the automatically generated finding and the result of the first interpretation finding to be set by a user operation. When the notification setting information input from the notification setting screen 561 is received from the interpretation terminal 5, the controller 41 stores (sets) the received notification setting information in the notification setting storage area 423 in association with the user ID of the user who is logging in.

That is, the controller 41 functions as a setting section capable of setting, by a user operation, which combination of the result of the automatically generated finding and the result of the interpretation finding is to be actively notified.

In a case where the second interpretation result and the image identification information are received from the interpretation terminal 5 by the data acquiring section 43, the controller 41 stores the received second interpretation result in the image database 422. The controller 41 also searches the image management table for a record having the acquired image identification information, and stores the acquired identification information of the second interpretation finding in the searched record. Further, the controller 41 updates the status information of the secondary interpretation of the medical image corresponding to the acquired image identification information to “interpreted (approved)”.

(Configuration of Interpretation Terminal)

The interpretation terminal 5 is, for example, a PACS client (PACS viewer). The interpretation terminal 5 is a display device that reads the medical image and the automatically generated finding from the image management apparatus 4 in response to an operation of an interpretation doctor who is a user, and displays them for interpretation. The user interprets the medical image on the interpretation terminal 5, and creates an interpretation finding (an interpretation report or the like) regarding the medical image.

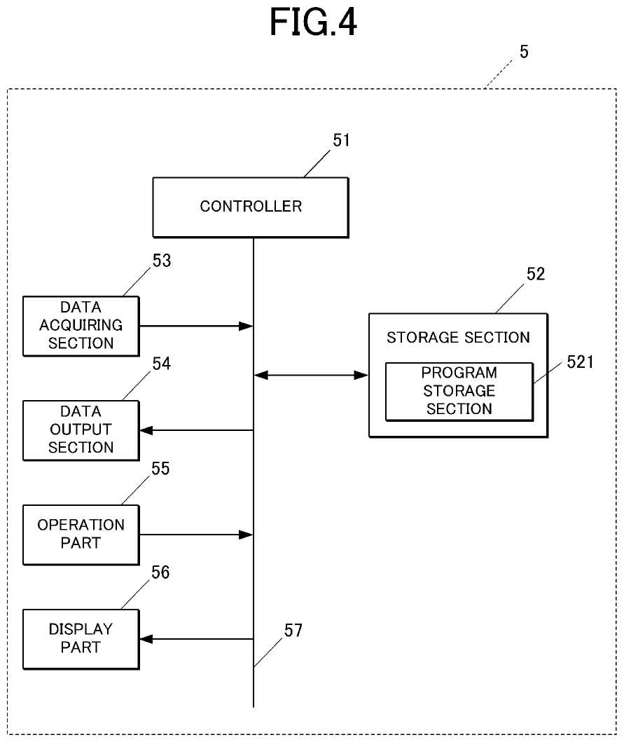

FIG. 4 is a block diagram showing a functional configuration of the interpretation terminal 5.

As illustrated in FIG. 4, the interpretation terminal 5 includes a controller (hardware processor) 51, a storage section 52, a data acquiring section 53, a data output section 54, an operation part 55, a display part 56, and the like. The respective units of the interpretation terminal 5 are connected by a bus 57.

The controller 51 is constituted by, e.g., a CPU and a RAM. The controller 51 integrally controls the operation of each unit of the interpretation terminal 5. Specifically, the CPU of the controller 51 reads various processing programs stored in a program storage section 521 of the storage section 52, loads the programs to a RAM, and executes various types of processing in accordance with the programs.

The storage section 52 is configured by a hard disk drive (HDD), a semiconductor memory, or the like. The storage section 52 includes a program storage section 521 that stores a program for the controller 51 to execute various processes, and the like. The storage section 52 also stores parameters, files, and the like required for execution of the programs stored in the program storage section 521.

The data acquiring section 53 is composed of, for example, a network interface. The data acquiring section 53 is configured to receive data from an external device connected via the in-hospital network N (and an external network) in a wired or wireless manner. Although the data acquiring section 53 is constituted by a network interface or the like in the present embodiment, the data acquiring section 53 may be constituted by a port or the like into which a USB memory, an SD card, or the like can be inserted.

The data output section 54 includes, for example, a network interface. The data output section 54 is configured to output data to an external device connected via the in-hospital network N (and an external network) in a wired or wireless manner. Note that in the present embodiment, the data output section 54 is assumed to be formed with a network interface and the like, but a connector for connecting to an external device, ports for various media such as a USB memory and the like are applicable.

The operation part 55 includes a keyboard having various keys, a pointing device such as a mouse, or a touch screen attached to the display part 56. The operation part 55 outputs, to the controller 51, an operation signal input by a key operation on the keyboard, a mouse operation, or a touch operation on the touch screen.

The display part 56 includes a monitor such as a liquid crystal display (LCD). The display part 56 displays various screens according to an instruction of a display signal input from the controller 51. Note that the number of monitors is not limited to one, and a plurality of monitors may be provided.

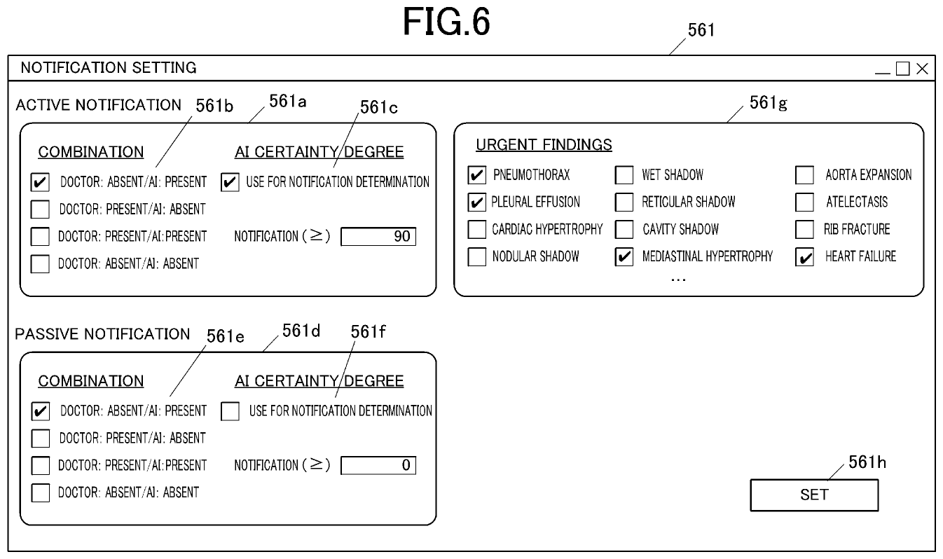

For example, when the display of the notification setting screen 561 (see FIG. 6) is instructed by the operation of the operation part 55 by the user, the controller 51 of the interpretation terminal 5 transmits a notification setting request to the image management apparatus 4 by the data output section 54. The user mentioned here is assumed to be a secondary interpretation doctor. The controller 51 causes the data acquiring section 53 to acquire the screen information on the notification setting screen 561 transmitted from the image management apparatus 4 and causes the display part 56 to display the screen information. When the notification setting information is input from the notification setting screen 561 by the operation of the operation part 55, the controller 51 transmits the input notification setting information to the image management apparatus 4 by the data output section 54.

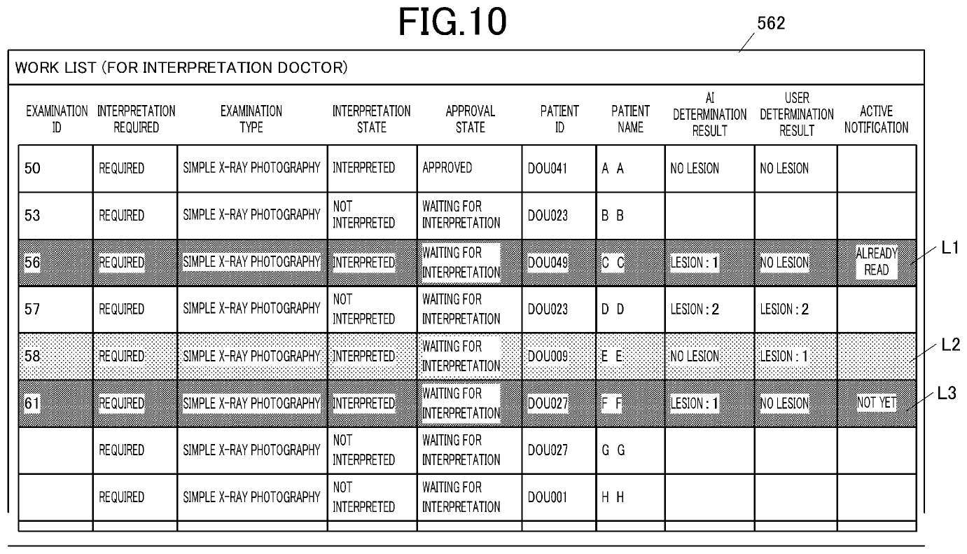

Further, for example, when the display of the examination list screen 562 (see FIG. 10) is instructed by the operation of the operation part 55, the controller 51 transmits the acquisition request of the examination list information to the image management apparatus 4 by the data output section 54. Then, the controller 51 causes the data acquiring section 53 to acquire the examination list information transmitted from the image management apparatus 4, and causes the display part 56 to display the examination list screen 562 on which the examination list information is displayed. At this time, the controller 51 allows the display part 56 to display, in different display manners, the information on the examination determined not to be a combination for active notification among the information on the examinations determined to be a combination for passive notification and the information on the medical image examination determined to be a combination for active notification.

For the information on the examination determined to be the combination for which the active notification is to be displayed, the controller 51 displays the information on the examination for which the display of the active notification is completed and the information on the examination of combination for which the display of the active notification is not completed on the display part 56 in different display manners.

In other words, the interpretation terminal 5 functions as a second external terminal.

Further, for example, when an active notification from the image management apparatus 4 is acquired by the data acquiring section 53, the controller 51 displays the active notification on the display part 56 in a pop-up manner, and transmits information indicating that the active notification has been received to the image management apparatus 4 by the data output section 54. When the OK button displayed in the pop-up of the active notification is pressed, the controller 51 displays all the information of the active notification, and transmits information indicating that the display of the active notification is completed to the image management apparatus 4 by the data output section 54.

(Configuration of External Terminal)

The external terminal 6 is a terminal device owned by a doctor. Examples of the external terminal 6 include a smartphone, a tablet terminal, a mobile phone, and a laptop computer, but the external terminal 6 is not limited thereto.

Upon receiving the active notification from the image management apparatus 4, the external terminal 6 performs push notification of the active notification and transmits, to the image management apparatus 4, information indicating that the active notification has been received. When the display of the active notification for which a push notification has been performed is pressed and opened, the external terminal 6 displays all the information of the active notification on the display part. Then, the external terminal 6 transmits information indicating that the display of the active notification is completed to the image management apparatus 4.

[Operation of Interpretation Management System 100]

Next, operations of notification setting and notification control in the interpretation management system 100 of the present embodiment will be described.

(Notification Setting Process)

Hereinafter, a notification setting process executed by the image management apparatus 4 will be described.

FIG. 5 is a flowchart showing the flow of the notification setting process executed by the controller 41 of the image management apparatus 4. The notification setting process is executed by the controller 41 in cooperation with the program stored in the program storage section 421, for example, when the data acquiring section 43 receives a notification setting request from the interpretation terminal 5.

First, the controller 41 outputs the screen information of the notification setting screen 561 to the interpretation terminal 5 by the data output section 44 to display the notification setting screen 561 on the interpretation terminal 5 (step S1), and waits for the reception of the notification setting information from the interpretation terminal (step S2).

FIG. 6 is an illustration of an example of the notification setting screen 561. The notification setting screen 561 is a screen for the user to set a combination to be actively notified among combinations of the result of the automatically generated finding and the result of the first interpretation finding.

As shown in FIG. 6, the notification setting screen 561 is provided with an active notification setting area 561a and a passive notification setting area 561d. The active notification setting area 561a is an area for setting a combination to be actively notified. The passive notification setting area 561d is an area for setting a combination to be passively notified.

The active notification setting area 561a is provided with a combination setting field 561b and an AI certainty degree setting field 561c.

The combination setting field 561b is a field for selecting a combination to be actively notified among combinations of the result of the automatically generated finding and the result of the first interpretation finding. In the combination setting field 561b, a combination selected (checked) from the following combinations (1) to (4) is set as a combination to be actively notified.
(1) A combination (doctor: absent/AI: present) in which the result of the doctor's finding (first interpretation finding) is absence of an abnormal finding and the result of the AI's finding (automatically generated finding) is presence of an abnormal finding
(2) A combination (doctor: present/AI: absent) in which the result of the doctor's findings (first interpretation findings) is the presence of abnormal findings and the result of the AI findings (automatically generated findings) is the absence of abnormal findings
(3) A combination (doctor: present/AI: present) in which the result of the finding of the doctor (first interpretation finding) is presence of an abnormal finding and the result of the finding of the AI (automatically generated finding) is presence of an abnormal finding;
(4) A combination (doctor: absent/AI: absent) in which the result of the doctor's finding (first interpretation finding) is absence of abnormal finding and the result of the AI's finding (automatically generated finding) is absence of abnormal finding

The AI certainty degree setting field 561c is a field for setting whether or not a certainty degree of a result of an automatically generated finding is further used as a condition for determining whether or not a combination is a combination for which active notification is performed. When the check box of “use for notification determination” is checked and the threshold value of the certainty degree is input, the input threshold value of the certainty degree is set as the threshold value used for determining whether or not the combination is to be actively notified.

The passive notification setting area 561d is provided with a combination setting field 561e and an AI certainty degree setting field 561f. Each setting field is obtained by replacing “active notification” in the description of the combination setting field 561b and the AI certainty degree setting field 561c with “passive notification”, and detailed description thereof will be omitted.

The notification setting screen 561 is provided with an urgent finding setting field 561g. The urgent finding setting field 561g is a selection field for selecting a target finding (an abnormal finding or a lesion with high urgency) to be actively notified in the case where the combination of the result of the automatically generated finding and the result of the first interpretation finding is a combination to be actively notified. When an abnormal finding is selected from the urgent finding setting field 561g, the selected abnormal finding is set as a finding to be actively notified.

For example, the user checks the urgent finding setting field 561g for an abnormal finding with high urgency that immediately requires treatment for pneumothorax, heart failure, or the like. Thus, in a case where a combination of the result of the automatically generated finding and the result of the first interpretation finding is a combination for which active notification is to be performed, setting can be made so that positive active notification is performed to a doctor if an abnormal finding extracted from the automatically generated finding is an abnormal finding with a high degree of urgency. Furthermore, the user does not check the urgent finding setting field 561g for an abnormal finding that is not highly urgent, such as a nodular shadow. Thus, in a case where the combination of the result of the automatically generated finding and the result of the first interpretation finding is a combination for which an active notification is performed, and in a case where an abnormal finding extracted from the automatically generated finding is an abnormal finding with low urgency, it is possible to make a setting so as to provide only a negative passive notification to a doctor.

In a case where there is no setting in the urgent finding setting field 561g, all combinations of the result of the automatically generated finding and the result of the first interpretation finding are targets of the active notification in a case where the combinations are combinations to be subjected to the active notification. Furthermore, without providing the urgent finding setting field 561g, in a case where a combination of the result of the automatically generated finding and the result of the first interpretation finding is a combination for which active notification is to be performed, all of the results may be subjected to active notification.

In the interpretation terminal 5, when input from the notification setting screen 561 is completed and a setting button 561h is pressed, the controller 51 causes the data output section 54 to transmit the input information as notification setting information to the image management apparatus 4.

In the present embodiment, it is assumed that the controller 41 of the image management apparatus 4 automatically sets a combination to be actively notified, which is set from the active notification setting area 561a of the notification setting screen 561, as a combination to be passively notified. However, the controller 41 may set only the combination set as the combination for the passive notification in the passive notification setting area 561d as the combination for the passive notification.

Furthermore, in the example of the notification setting screen 561 illustrated in FIG. 6, the user can set both a combination for which an active notification is performed and a combination for which a passive notification is performed, of the result of the automatically generated finding and the result of the first interpretation finding. However, a configuration may be adopted in which the user can input only a combination of active notifications, and the controller 41 may automatically set a combination for which active notification is not set as a combination for which passive notification is performed. If the controller 41 determines that the combination is not a combination to be actively notified, the controller 41 may passively notify the interpretation terminal 5 of predetermined information without actively notifying the interpretation terminal 5 of the predetermined information.

When the notification setting information from the interpretation terminal 5 is obtained by the data acquiring section 43 (Step S2; YES), the controller 41 stores (sets) the received notification setting information in the notification setting storage area 423 in association with the user IDs of the users who are logging in (Step S3). The controller 41 then ends the notification setting process.

With the notification setting process, the controller 41 can set, according to a user operation, active notification for a combination in which the result of the automatically generated finding has an abnormal finding and the result of the first interpretation finding does not have an abnormal finding among combinations of the result of the automatically generated finding and the result of the first interpretation finding.

By this setting, when there is a possibility of oversight on the doctor side, it is possible to quickly notify the doctor.

Further, by the notification setting process, the controller 41 can set not to perform the active notification for a combination in which the result of the automatically generated finding has an abnormal finding and the result of the first interpretation finding does not have an abnormal finding among combinations of the result of the automatically generated finding and the result of the first interpretation finding according to a user operation.

With this setting, it is possible to improve interpretation efficiency by not performing active notification when there is a possibility of a false positive. In this case, it is preferable to set the combination as a combination for which the passive notification is performed.

Further, by the notification setting process, the controller 41 can set not to perform the active notification for a combination in which the result of the automatically generated finding does not have an abnormal finding and the result of the first interpretation finding does not have an abnormal finding among combinations of the result of the automatically generated finding and the result of the first interpretation finding according to a user operation.

By this setting, it is possible to improve interpretation efficiency by not performing active notification in a case where there is a high probability that an abnormality (lesion) is not present. In this case, it is preferable to set the combination as a combination for which the passive notification is performed.

In the notification setting process, the controller 41 can set to perform the active notification or the passive notification for a combination in which the result of the automatically generated finding has an abnormal finding and the result of the first interpretation finding has an abnormal finding among combinations of the result of the automatically generated finding and the result of the interpretation finding according to a user operation.

By setting active notification for a combination in which the result of the automatically generated finding is an abnormal finding and the result of the first interpretation finding is an abnormal finding, it is possible to actively notify a combination in which the probability of the presence of an abnormality (lesion) is high. Therefore, it is possible to shorten the time until diagnosis decision by the medical specialist. Furthermore, by setting to provide a passive notification for a combination in which the result of the automatically generated finding has an abnormal finding and the result of the first interpretation finding has an abnormal finding, it is possible to provide a notification that the primary interpretation doctor has already detected a lesion. Therefore, it is possible to improve efficiency of interpretation (secondary interpretation).

That is, by the notification setting process, it is possible to set the combination to be actively notified among the combinations of the result of the automatically generated finding and the result of the first interpretation finding according to the needs of the user such as the secondary interpretation doctor.

(Notification Control Process)

Next, a notification control process executed by the image management apparatus 4 will be described.

FIG. 7 is a flowchart showing a flow of a notification control process executed by the controller 41 of the image management apparatus 4. The notification control process is executed by the controller 41 in cooperation with the program stored in the program storage section 421, for example, when the data acquiring section 43 receives the first interpretation finding from the interpretation terminal 5.

First, the controller 41 acquires the received first interpretation finding (step S11).

Then, the controller 41 acquires the automatically generated finding of the medical image corresponding to the acquired first interpretation finding (step S12).

For example, the controller 41 searches the image management table of the image database 422 for a record having the image identification information received together with the first interpretation finding. Then, the controller 41 specifies the identification information of the automatically generated finding corresponding to the first interpretation finding based on the searched record, and acquires the automatically generated finding of the specified identification information from the image database 422.

Next, the controller 41 determines whether or not the combination of the result of the automatically generated finding and the result of the first interpretation finding is a combination for active notification (step S13).

In step S13, first, the controller 41 refers to the record searched in step S12, and specifies a secondary interpretation doctor who interprets the medical image corresponding to the acquired first interpretation finding. Next, the controller 41 reads out notification setting information corresponding to the identified secondary interpretation doctor from the notification setting storage area 423. Based on the read notification setting information, the controller 41 determines whether the combination of the acquired result of the automatically generated finding and the result of the first interpretation finding is the set combination to be actively notified.

For example, when the combination of the result of the presence or absence of an abnormal finding in the automatically generated finding and the result of the presence or absence of an abnormal finding in the first interpretation finding is a combination set as a combination to be actively notified, the controller 41 determines that the combination is a combination to be actively notified. In a case where the above combination is not the combination set as the combination to be actively notified, the controller 41 determines that the above combination is not the combination to be actively notified. The presence or absence of an abnormal finding is synonymous with the presence or absence of lesion detection.

When the use of the AI certainty degree for notification determination and a threshold value of the certainty degree are set in the notification setting information, the controller 41 makes the determination as follows. In a case where the combination of the result of the presence or absence of the abnormal finding in the automatically generated finding and the result of the presence or absence of the abnormal finding in the first interpretation finding is the combination set as the combination to be actively notified and the certainty degree for the automatically generated finding is equal to or greater than the set threshold value, it is determined that the combination is the combination to be actively notified. Otherwise, it is determined that the combination is not to be actively notified. In the notification setting screen 561, the threshold value of the certainty degree for not performing the active notification may be set by the user. Then, the controller 41 may determine that the combination is not to be actively notified when the certainty degree of the automatically generated finding is equal to or less than the set threshold value, and otherwise determine that the combination is to be actively notified.

Here, the automatically generated finding is structured data obtained by computer processing (AI analysis). On the other hand, the interpretation finding is interpretation report created by a doctor, for example, and is not limited to a structured finding. For example, in the interpretation finding, the presence or absence of an abnormal finding is also expressed by “+” or “−” in some cases, and is expressed by a character string indicating the presence or absence, such as “presence” or “absence” of an abnormality, or “recognized” or “not recognized” of an abnormality in some cases. That is, it is conceivable that there is a variation in expression of interpretation findings. For this reason, the controller 41 may have a function of structuring the first interpretation finding as data (structured data) including a character string or the like which can be collated (compared) with the automatically generated finding as a precondition for matching the automatically generated finding with the first interpretation finding. In this case, for example, the storage section 42 is caused to store dictionary data or the like which is used for generating structured data and which defines correspondences between character strings and the like, and the controller 41 refers to this dictionary data and structures the first interpretation finding.

Note that even in a case where there is a slight difference in expression between the “automatically generated finding” and the “interpretation finding”, when it is possible to determine coincidence/non-coincidence of both of them, the controller 41 may collate (compare) the “automatically generated finding” and the “interpretation finding” without structuring the “interpretation finding”.

For example, the controller 41 can associate expressions such as “+”, “presence” of an abnormality, and abnormality is “recognized” with the same meaning as “information indicating that there is an abnormal finding (+)” extracted from the “automatically generated finding”. Further, the controller 41 can associate the expressions such as “−”, “absence” of abnormality, and abnormality is “not recognized” with the same meaning as the “information indicating that there is no abnormal finding (−)” extracted from the “automatically generated finding”. In such a case, the controller 41 may collate (compare) the “automatically generated finding” with the “interpretation finding” without structuring the “interpretation finding”.

If it is determined that the combination of the result of the automatically generated finding and the result of the first interpretation finding is a combination for active notification (YES in step S13), the controller 41 determines whether an abnormal finding with high urgency is set in the notification setting information read out in step S13 (step S14).

In a case where it is determined that an abnormal finding with high urgency is not set (step S14; NO), the controller 41 proceeds to step S16.

When determining that an abnormal finding with high urgency is set (step S14; YES), the controller 41 determines whether an abnormal finding is extracted from the automatically generated findings. Next, upon determination that an abnormal finding has been extracted from the automatically generated findings, the controller 41 determines whether the abnormal finding extracted from the automatically generated findings is a set abnormal finding with high urgency (step S15).

If the controller 41 determines that the abnormal finding extracted from the automatically generated finding is the set abnormal finding with high urgency (YES in step S15), the controller 41 shifts to step S16.

When determining that the abnormal finding extracted from the automatically generated finding is not the set finding with high urgency (step S15; NO), the controller 41 proceeds to step S24.

In Step S16, the controller 41 sets the active notification flag and the passive notification flag of the record having the image identification information received together with the first interpretation finding from the interpretation terminal 5 in the image management table of the image database 422 to ON. Next, the controller 41 sets the status information of the active notification to “not yet” (step S16). Then, the controller 41 actively notifies the notification destinations (first external terminals) of predetermined information (step S17).

In step S17, the controller 41 actively notifies the notification destination (first external terminal) corresponding to the secondary interpretation doctor specified in step S12, which is stored in the notification destination storage area 424, of predetermined information. To be specific, the controller 41 transmits the predetermined information to the notification destination corresponding to the secondary interpretation doctor specified in step S12 by the data output section 44. As the notification destination corresponding to the secondary interpretation doctor, for example, the interpretation terminal 5 or the external terminal 6 used by the secondary interpretation doctor is set.

Examples of the predetermined information to be actively notified include the following. Information such as a message corresponding to a combination of the result of the presence or absence of an abnormal finding in the automatically generated finding and the result of the presence or absence of an abnormal finding in the first interpretation finding, the examination ID of the target examination, the patient ID, the patient name, the lesion name, the presence or absence of an abnormal finding in the automatically generated finding, and the presence or absence of an abnormal finding in the interpretation finding. Examples of the above-mentioned message include the following. “A case that may be overlooked has been detected. Please check.” “A case with a high probability of being abnormal has been detected. Please check”. “There is a discrepancy between the automatically generated finding and the interpretation finding”, and the like.

Furthermore, it is preferable that the active notification includes information indicating that the notification is based on the result of the first interpretation finding. Accordingly, the secondary interpretation doctor who confirms the active notification on the first external terminal of the notification destination can recognize that the notification is performed based on the result of the confirmation of the medical image by the primary interpretation doctor. It is preferable that the active notification includes information indicating that the notification is based on the result of the automatically generated finding and the result of the first interpretation finding. Accordingly, the secondary interpretation doctor who confirms the active notification on the first external terminal of the notification destination can recognize that the notification is performed based on the confirmation result of the medical image by the primary interpretation doctor and the analysis result of the medical image by the computer processing.

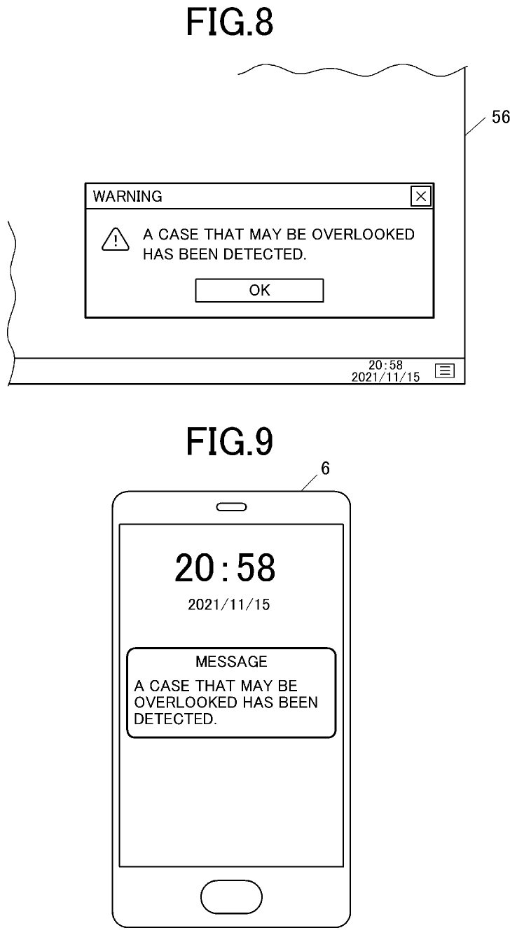

When the predetermined information actively notified from the image management apparatus 4 is received, the first external terminal of the notification destination notifies the received predetermined information. For example, as shown in FIG. 8, the interpretation terminal 5 of the notification destination displays at least a part of the received information on the display part 56 in a pop-up manner. Furthermore, for example, as illustrated in FIG. 9, the external terminal 6 ((a smartphone, a tablet terminal, or the like) that is a notification destination performs a push notification (displays on a display part) of at least a part of the received information. Then, the first external terminal transmits information indicating that the active notification has been received to the image management apparatus 4.

Furthermore, in the interpretation terminal 5, for example, when an OK button in a pop-up display is pressed, the interpretation terminal 5 displays the received predetermined information (all information) on the display part, and transmits information indicating that the display of the active notification has been completed to the image management apparatus 4. For example, when the display of the push notification is tapped on the external terminal 6, the external terminal 6 displays the received predetermined information (all information) on the display part and transmits information indicating that the display of the active notification is completed to the image management apparatus 4.

In the image management apparatus 4, when the active notification to the notification destination is completed, the controller 41 determines, with the data acquiring section 43, whether or not information indicating that the active notification has been received from the notification destination has been received (step S18).

When it is determined that the information indicating that the active notification has been received has not been received from the notification destination (step S18; NO), the controller 41 proceeds to step S20.

When determining that the information indicating that the active notification has been received from the notification destination has been received (step S18; YES), the controller 41 updates, to “notified”, the active notification status information of the record having the image identification information received together with the first interpretation finding in the image management table (step S19). Then, the controller 41 proceeds to step S20.

In step S20, the controller 41 determines whether or not information indicating that the display of the active notification is completed has been received from the notification destination by the data acquiring section 43 (step S20).

When it is determined that the information indicating that the display of the active notification has been completed is not received from the notification destination (step S20; NO), the controller 41 proceeds to step S22.

When it is determined that the information indicating that the display of the active notification is completed is received from the notification destination (step S20; YES), the controller 41 updates the active notification status information of the record having the image identification information received together with the first interpretation finding in the image management table to “already read” (step S21). Then, the controller 41 proceeds to step S22.

In step S22, the controller 41 determines whether or not a request to acquire examination list information has been received from the interpretation terminal 5 by the data acquiring section 43 (step S22).

When it is determined that the acquisition request of the examination list information has not been received (step S22; NO), the controller 41 returns to step S18.

If it is determined that the request to acquire the examination list information has been received from the interpretation terminal 5 (YES in step S22), the controller 41 transmits the examination list information to the interpretation terminal 5 by the data output section to perform passive notification (step S23), and ends the notification control process.

In step S23, the controller 41 extracts, for example, a record that matches a predetermined condition from the image management table of the image database 422. Then, the controller 41 generates examination list information including predetermined items of the extracted record, and transmits the examination list information to the interpretation terminal 5 as the second external terminal by the data output section 44. Examples of the record matching the predetermined condition include a record in which the examination date and time is within a predetermined period, a record in which the type of modality is a predetermined type, and a record in which the secondary interpretation status information is not “interpretation completed”. At this time, the controller 41 includes information on predetermined items to be passively notified, such as the examination ID, the patient ID, the patient name, the AI determination result, and the user result, in the examination list information, and also transmits information on items of the active notification flag, the passive notification flag, and the status information of the active notification. Thus, predetermined information (an examination ID, a patient ID, a patient name, an AI determination result, a user result, and the like) about an examination for which the combination of the result of the automatically generated finding and the result of the first interpretation finding is a combination to be passively notified is passively notified.

In the interpretation terminal 5, in a case where the data acquiring section 53 receives the predetermined information of which the passive notification has been performed, the controller 51 displays the examination list screen 562 based on the received examination list information on the display part 56.

At this time, the controller 51 displays the information of the examination in which the active notification flag is ON in a first color, and displays the information of the examination in which the active notification flag is OFF and the passive notification flag is ON in a second color different from the first color. Thus, the controller 51 displays, on the display part 56, the predetermined information to be passively notified when it is determined that the combination of the result of the automatically generated finding and the result of the first interpretation finding is a combination to be actively notified, and the predetermined information to be passively notified when it is determined that the combination is not a combination to be actively notified, in different display manners.

The controller 51 displays the content (“not yet”, “notified”, or “already read”) of the active notification status information for the information of the examination in which the active notification flag is ON. As a result, the controller 51 displays, on the display part 56, the combination for which the display of the active notification on the first external terminal of the notification destination is completed and the combination for which the display of the active notification on the terminal of the notification destination is not completed in different display manners among the combinations for which the active notification is performed.

FIG. 10 is a view illustrating an example of an examination list screen 562 displayed on the interpretation terminal 5. FIG. 10 shows a display example of the examination list screen 562 in a case of the following combination. A case where a combination in which the result of the automatically generated finding is the presence of the abnormal finding and the result of the first interpretation finding is the absence of the abnormal finding is a combination to be actively notified, and a combination in which the result of the automatically generated finding is the absence of the abnormal finding and the result of the first interpretation finding is the presence of the abnormal finding is a combination to be passively notified without active notification.

As shown in FIG. 10, in the examination list screen 562, the information (L1, L3 in FIG. 10) of the examination in which the combination of the result of the automatically generated finding and the result of the first interpretation finding is the combination to be actively notified is displayed in dark gray. In addition, the information (L2 in FIG. 10) of the examination which is the combination of the passive notification and is not the combination of the active notification is displayed in light gray. Furthermore, as the information on the examination of the combination to be actively notified, “already read” is displayed when the display of the active notification is completed, and “not yet” or “notified” is displayed for the information on the examination for which the display of the active notification is not completed.

As described above, the display manner of the predetermined information to be passively notified when it is determined that the combination is to be actively notified and the display manner of the predetermined information to be passively notified when it is determined that the combination is not to be actively notified are displayed on the display part 56 in different display manners. Therefore, the user can easily grasp the examination of the medical image of the combination for which the active notification is performed and the examination of the medical image of the combination for which only the passive notification is performed. In addition, it is possible to easily distinguish between the information on the combination in which the display of the active notification is completed and the information on the combination in which the display of the active notification is not completed among the combinations to be subjected to the active notification.

On the other hand, in a case in which it is determined that the combination of the result of the automatically generated finding and the result of the first interpretation finding is not the combination for active notification (Step S13; NO), the controller 41 determines whether or not the combination of the result of the automatically generated finding and the result of the first interpretation finding is the combination for passive notification (Step S24).

In step S24, the controller 41 determines, based on the notification setting information corresponding to the identified secondary interpretation doctor, whether the combination of the result of the automatically generated finding and the result of the first interpretation finding is a combination to be passively notified. The description of an example of a specific determination method is the same as the description of the determination method of the combination of the active notifications in step S13 in which the active notification is replaced with the passive notification, and thus the description thereof will be omitted.

If it is determined that the combination of the result of the automatically generated finding and the result of the first interpretation finding is a combination to be passively notified (YES in step S24), the controller 41 sets the passive notification flag of the record having the image identification information received together with the first interpretation finding in the image management table to ON (step S25).

Next, the controller 41 waits for the data acquiring section 43 to receive a request to acquire examination list information from the interpretation terminal 5 (step S26).

In a case where the acquisition request for the examination list information is received from the interpretation terminal 5 (step S26; YES), the controller 41 transmits the examination list information to the interpretation terminal 5 using the data output section 44 to perform the passive notification (step S27). The controller 41 then ends the notification control process.

Since the process of step S27 is the same as the process of step S23, the description thereof will be omitted.

In the interpretation terminal 5, in a case where the examination list information is received by the data acquiring section 53, the controller 51 displays an examination list screen 562 (refer to FIG. 10) based on the received examination list information on the display part 56.

On the other hand, in a case in which it is determined in Step S24 that the combination of the result of the automatically generated finding and the result of the first interpretation finding is not the combination for the passive notification (Step S24; NO), the controller 41 ends the notification control process.

As described above, the controller 41 of the image management apparatus 4 acquires an automatically generated finding obtained by computer processing on a medical image in cooperation with a program stored in the program storage section 421. Further, the controller 41 acquires the first interpretation finding created by the user on the basis of the medical image. Then, the controller 41 determines whether or not the combination of the acquired result of the automatically generated finding and the result of the first interpretation finding is a combination to be actively notified, and actively notifies predetermined information when determining that the combination is a combination to be actively notified.

Therefore, in a case where the result of the automatically generated finding obtained by the computer processing and the result of the interpretation finding by the doctor are a combination to be actively notified, it is possible to quickly and reliably notify the doctor that the combination of the result of the automatically generated finding and the result of the interpretation finding by the doctor is the combination to be actively notified even if the doctor does not try to display the combination by himself/herself.

According to the present embodiment, an active notification can be made when a combination of an automatically generated finding obtained by computer processing and a doctor's interpretation finding is a combination for which an active notification should be made.

For example, the controller 41 can set, by a user operation, a combination to be actively notified among combinations of the result of the automatically generated finding and the result of the first interpretation finding. Then, the controller 41 determines whether or not the combination of the result of the automatically generated finding and the result of the first interpretation finding is a combination to be actively notified based on the set combination to be actively notified.

Therefore, when the combination of the result of the automatically generated finding and the result of the first interpretation finding is a combination for which the user desires an active notification, the active notification can be performed quickly and reliably.

Furthermore, for example, the controller 41 can set, by a user operation, a combination to be actively notified among combinations of the result of the automatically generated finding and the result of the first interpretation finding, and a threshold value of the certainty degree of the automatically generated finding. Then, the controller 41 determines whether the combination of the result of the automatically generated finding and the result of the first interpretation finding is a combination to be actively notified, based on the set combination to be actively notified and the threshold value of the certainty degree of the automatically generated finding.

Therefore, when the combination of the result of the automatically generated finding and the result of the first interpretation finding is a combination and a certainty degree for which the user desires an active notification, the active notification can be performed.

Note that the present invention is not limited to the above-described embodiment, and various modifications can be made without departing from the spirit and scope of the present invention.

For example, in the above-described embodiment, a case where the controller 41 of the image management apparatus 4 realizes the function of the interpretation management device according to the embodiment of the present invention has been described as an example. However, a controller of another apparatus such as the analysis apparatus 3 or the interpretation terminal 5 may realize the function of the interpretation management apparatus according to the embodiment of the present invention.

For example, the functions of the present invention may be realized by cooperation between the controller 31 of the analysis apparatus 3 and the program stored in the program storage section 321. Alternatively, the functions of the present invention may be realized by cooperation between the controller 51 of the interpretation terminal 5 and the program stored in the program storage section 521. Alternatively, the functions of the present invention may be realized by cooperation between a controller of another terminal and a program stored in a storage section.

For example, in the case of a configuration in which the functions of the present invention are realized by the analysis apparatus 3, a program(s) for executing the notification setting process and the notification control process is stored in the program storage section 321 of the storage section 32 of the analysis apparatus 3. Furthermore, the storage section 32 is provided with areas similar to the above-described notification setting storage area 423 and notification destination storage area 424, and stores a table similar to the interpretation doctor table 425, user information, and the like. Furthermore, the storage section 32 stores automatically generated findings generated based on a medical image in association with accompanying information of the medical image.

When the notification setting request is input to the interpretation terminal 5, the interpretation terminal 5 accesses the analysis apparatus 3 and transmits the notification setting request. In the analysis apparatus 3, in a case where the data acquiring section 33 receives the notification setting request from the interpretation terminal 5, the controller 31 executes notification setting process. The notification setting process is the same process as the processing described with reference to FIG. 5. The notification setting information is stored in the notification setting storage area of the storage section 32.

The controller 31 executes the notification control process at a predetermined timing. For example, when the image management apparatus 4 receives the first interpretation finding and the image identification information, the first interpretation finding and the image identification information are transmitted from the image management apparatus 4 to the analysis apparatus 3. Upon receiving the first interpretation finding and the image identification information from the image management apparatus 4, the controller 31 executes the notification control process. The notification control process is the same as the process described with reference to FIG. 7. Provided that in a case where the analysis apparatus 3 and the image management apparatus 4 are separate from each other, turning on of the active notification flag, turning on of the passive notification flag, and updating of the status information on the active notification are performed by instructing the image management apparatus 4 via the in-hospital network N. The passive notification is performed by setting a passive notification flag in the image management table of the image management apparatus 4 to ON. The transmission of the examination list information reflecting the passive notification is performed by the image management apparatus 4. Alternatively, a configuration may be adopted in which a table similar to the image management table of the image database 422 is provided in the storage section 32 and the controller 31 can display the examination list information on the display part 36 in response to a request from the operation part 35, and in which the passive notification is performed by displaying the examination list information including an item of predetermined information on the display part 36.

In addition, for example, in a case of a configuration in which the function of the present invention is realized in the interpretation terminal 5, a program(s) for executing the notification setting process and the notification control process is stored in the program storage section 521 of the storage section 52 of the interpretation terminal 5. Further, the storage section 52 is performed with storage areas similar to the notification setting storage area 423 and the notification destination storage area 424 described above, and stores tables similar to the interpretation doctor table 425, user information, and the like.

In the interpretation terminal 5, when a notification setting request is input by the operation part 55, the controller 51 executes notification setting process. The notification setting process is the same process as the processing described with reference to FIG. 5. The notification setting information is stored in the notification setting storage area of the storage section 52.

Furthermore, upon input of the first interpretation finding, the controller 51 executes notification control process. The notification control process is the same as the process described with reference to FIG. 7. Note that turning on of the active notification flag, turning on of the passive notification flag, and updating of the status information of the active notification are performed by instructing the image management apparatus 4 via the in-hospital network N. The passive notification is performed by setting the passive notification flag of the image management table of the image management apparatus 4 to ON, receiving the examination list information reflecting the passive notification in response to the acquisition request of the examination list information by the operation part 55, and displaying the examination list information on the display part 56.

In the above-described embodiment, the analysis apparatus 3, the image management apparatus 4, and the interpretation terminal 5 are illustrated as independent apparatuses in FIG. 1. However, the image management apparatus 4 and the interpretation terminal 5, the analysis apparatus 3 and the image management apparatus 4, and the analysis apparatus 3, the image management apparatus 4, and the interpretation terminal 5 may be configured as a single apparatus.

The functions of the interpretation management system according to the present invention may be distributed to hardware processors of a plurality of apparatuses.

In the above-described embodiment, a case where the result of the automatically generated finding is the result of the presence or absence of the detection of the lesion of the automatically generated finding and the result of the first interpretation finding is the result of the presence or absence of the detection of the lesion of the first interpretation finding has been described as an example, but the present invention is not limited thereto. For example, the result of the automatically generated finding may be the result of the detected position of the lesion of the automatically generated finding, and the result of the first interpretation finding may be the result of the detected position of the lesion of the first interpretation finding. In this case, for example, among combinations of the result of whether or not a lesion is detected in the automatically generated finding and the result of the detected position of a lesion in the first interpretation finding, a combination with a difference or a combination with no difference is set as a combination for which active notification is performed or a combination for which passive notification is performed. Thus, active notification or passive notification can be executed according to the setting.

Furthermore, although the case where notification setting is performed for each secondary interpretation doctor (each user) has been described as an example in the above embodiment, notification setting may be performed for each medical facility or for each group in a medical facility. Furthermore, a notification destination may also be set for each medical facility or for each group in a medical facility.

Although it has been described in the aforementioned embodiment that the passive notification is displayed on the examination list screen, the present invention is not limited thereto, and the passive notification may be displayed on another screen.

In the above-described embodiment, it has been described that the examination list screen (examination list information) is displayed on the interpretation terminal 5 and the passive notification is notified on the examination list screen of the interpretation terminal 5. However, other devices such as the analysis apparatus 3, the image management apparatus 4, and the external terminal 6 may be configured to be able to display the examination list screen, and the passive notification may be notified on the examination list screen of these devices. In addition, these apparatuses may implement the function as the second external terminal.

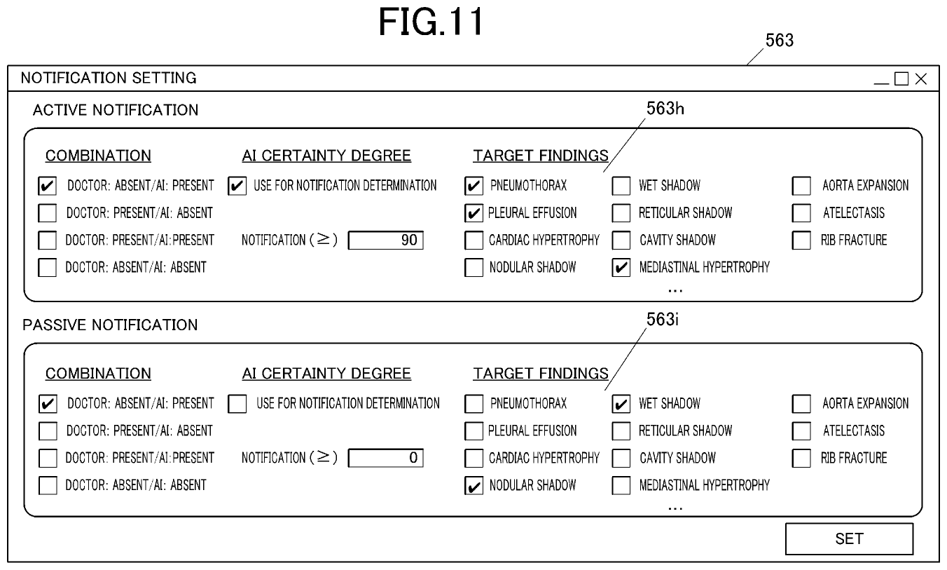

Further, the notification setting screen may be provided with a target finding setting field 563h for the user to set a target finding to be actively notified and a target finding setting field 563i for the user to set a target finding to be passively notified, as in a notification setting screen 563 illustrated in FIG. 11. The target finding is, for example, the type of lesion. Then, in a case in which the result of the automatically generated finding and the result of the first interpretation finding are a combination for active notification and the detected lesion is a lesion set as the target finding, the controller 41 may perform active notification. Alternatively, the controller 41 may perform active notification when the result of the automatically generated finding and the result of the first interpretation finding are a combination for which active notification is to be performed, the certainty degree is equal to or more than a predetermined threshold value or exceeds a predetermined threshold value, and the detected lesion is a lesion set as a target finding. Further, the controller 41 may perform the passive notification in a case where the result of the automatically generated finding and the result of the first interpretation finding are a combination to be passively notified and the detected lesion is a lesion set as the target finding. Alternatively, the controller 41 may perform a passive notification in a case where the combination of the result of the automatically generated finding and the result of the first interpretation finding is a combination for which a passive notification is performed, the certainty degree is equal to or more than a predetermined threshold value or exceeds a predetermined threshold value, and the detected lesion is a lesion set as a target finding.

Accordingly, even if the combination of the result of the automatically generated finding and the result of the first interpretation finding is the same, it is possible to change whether to perform the active notification or the passive notification depending on the type of lesion.

In the above-described embodiment, the case where the medical information according to the embodiment of the present invention is the medical image has been described as an example, but the medical information is not limited to the medical image.

Information acquired by various examinations on a patient and the like may be broadly included in the medical information. For example, results obtained by various examinations or the like such as electrocardiogram waveform data, cardiac sound data, and data related to blood flow may also be included in the medical information.

Further, in the above description, an example in which a hard disk, a semiconductor nonvolatile memory, or the like is used as a computer-readable medium of the program according to the present invention has been disclosed, but the present invention is not limited to this example. As other computer-readable media, portable recording media such as CD-ROMs can be applied. Furthermore, a carrier wave is also applied as a medium for providing data of the program according to the present invention via a communication line.

In addition, the detailed configuration and detailed operation of each apparatus constituting the interpretation management system can be appropriately changed without departing from the scope of the invention.

Although embodiments of the present invention have been described and illustrated in detail, the disclosed embodiments are made for purposes of illustration and example only and not limitation. The scope of the present invention should be interpreted by terms of the appended claims.

1. An interpretation management apparatus comprising a hardware processor that:
acquires an automatically generated finding obtained by computer processing on medical information;
acquires a first interpretation finding created by a user based on the medical information;
determines whether or not a result combination is a combination for which an active notification is to be performed, the result combination being a combination of a result of the automatically generated finding and a result of the first interpretation finding; and
actively notifies predetermined information in response to determination that the result combination is the combination for which the active notification is to be performed.

2. The interpretation management apparatus according to claim 1, wherein
the hardware processor enables setting, by a user operation, the combination for which the active notification is to be performed among the result combination, and
the hardware processor determines, based on the set combination for which the active notification is to be performed, whether or not the result combination is the combination for which the active notification is to be performed.

3. The interpretation management apparatus according to claim 1, wherein
the hardware processor enables setting, by a user operation, the combination for which the active notification is to be performed among the result combination and a threshold value of a certainty degree of the automatically generated finding, and
the hardware processor determines, based on the set combination for which the active notification is to be performed and the threshold value of the certainty degree of the automatically generated finding, whether or not the result combination is the combination for which the active notification is to be performed.

4. The interpretation management apparatus according to claim 1, wherein the hardware processor actively notifies that the active notification is a notification based on the result of the first interpretation finding.

5. The interpretation management apparatus according to claim 1, wherein the hardware processor actively notifies that the active notification is a notification based on the result of the automatically generated finding and the result of the first interpretation finding.

6. The interpretation management apparatus according to claim 1, wherein the result of the automatically generated finding is a result of presence or absence of detection of a lesion in the automatically generated finding, and the result of the first interpretation finding is a result of presence or absence of detection of a lesion in the first interpretation finding.

7. The interpretation management apparatus according to claim 1, wherein the result of the automatically generated finding is a result of a detected position of a lesion in the automatically generated finding, and the result of the first interpretation finding is a result of a detected position of a lesion in the first interpretation finding.

8. The interpretation management apparatus according to claim 2, wherein the hardware processor enables setting, by a user operation, to perform the active notification for a combination in which the result of the automatically generated finding has an abnormal finding and the result of the first interpretation finding does not have an abnormal finding among the result combination.

9. The interpretation management apparatus according to claim 2, wherein the hardware processor enables setting, by a user operation, not to perform the active notification for a combination in which the result of the automatically generated finding has an abnormal finding and the result of the first interpretation finding does not have an abnormal finding among the result combination.

10. The interpretation management apparatus according to claim 2, wherein the hardware processor enables setting, by a user operation, not to perform the active notification for a combination in which the result of the automatically generated finding does not have an abnormal finding and the result of the first interpretation finding does not have an abnormal finding among the result combination.

11. The interpretation management apparatus according to claim 2, wherein the hardware processor enables setting, by a user operation, to perform the active notification or a passive notification for a combination in which the result of the automatically generated finding has an abnormal finding and the result of the first interpretation finding has an abnormal finding among the result combination.

12. The interpretation management apparatus according to claim 2, wherein the hardware processor actively notifies a user in response to an urgency of an abnormal finding extracted from the automatically generated findings being high.

13. The interpretation management apparatus according to claim 3, wherein the hardware processor determines that the result combination is the combination for which the active notification is to be performed in response to the result combination being the set combination for which the active notification is to be performed and the certainty degree of the automatically generated finding being equal to or larger than the threshold value.

14. The interpretation management apparatus according to claim 3, wherein the hardware processor determines that the result combination is a combination for which the active notification is not to be performed in response to the certainty degree of the automatically generated finding being equal to or smaller than the threshold value.

15. The interpretation management apparatus according to claim 1, wherein the hardware processor
actively notifies a first external terminal of the predetermined information in response to the determination that the result combination is the combination for which the active notification is to be performed, and
does not actively notify the first external terminal of the predetermined information and passively notifies a second external terminal of predetermined information in response to determination that the result combination is not the combination for which the active notification is to be performed.

16. An interpretation management system comprising:
the interpretation management apparatus according to claim 15, and
a second external terminal connected to the interpretation management apparatus via a network, wherein
the hardware processor passively notifies the second external terminal of predetermined information in response to the determination that the result combination is the combination for which the active notification is to be performed, and
a hardware processor of the second external terminal displays, on a display part of the second external terminal in different display manners, the predetermined information that is passively notified to the second external terminal in response to the determination that the result combination is not the combination for which the active notification is to be performed and the predetermined information that is passively notified to the second external terminal in response to the determination that the result combination is the combination for which the active notification is to be performed.

17. An interpretation management system comprising:
the interpretation management apparatus according to claim 15, and
a second external terminal connected to the interpretation management apparatus via a network, wherein
the hardware processor passively notifies the second external terminal of predetermined information in response to the determination that the result combination is the combination for which the active notification is to be performed, and
a hardware processor of the second external terminal displays, on a display part of the second external terminal in different display manners, a combination for which displaying of the active notification on the first external terminal is completed and a combination for which displaying of the active notification on the first external terminal is not completed among the combination for which the active notification is to be performed to the first external terminal.

18. An interpretation management system comprising a hardware processor that:
acquires an automatically generated finding obtained by computer processing on medical information;
acquires a first interpretation finding created by a user based on the medical information;
determines whether or not a result combination is a combination for which an active notification is to be performed, the result combination being a combination of a result of the automatically generated finding and a result of the first interpretation finding; and
actively notifies predetermined information in response to determination that the result combination is the combination for which the active notification is to be performed.

19. An image management apparatus comprising a hardware processor that:
acquires an automatically generated finding obtained by computer processing on medical information;
acquires a first interpretation finding created by a user based on the medical information;
determines whether or not a result combination is a combination for which an active notification is to be performed, the result combination being a combination of a result of the automatically generated finding and a result of the first interpretation finding; and
actively notifies predetermined information in response to determination that the result combination is the combination for which the active notification is to be performed.

20. An analysis apparatus comprising a hardware processor that:
acquires an automatically generated finding obtained by computer processing on medical information;
acquires a first interpretation finding created by a user based on the medical information;
determines whether or not a result combination is a combination for which an active notification is to be performed, the result combination being a combination of a result of the automatically generated finding and a result of the first interpretation finding; and
actively notifies predetermined information in response to determination that the result combination is the combination for which the active notification is to be performed.

21. An interpretation management method comprising:
first acquiring that is acquiring an automatically generated finding obtained by computer processing on medical information;
second acquiring that is acquiring a first interpretation finding created by a user based on the medical information;
determining that is determining whether or not a result combination is a combination for which an active notification is to be performed, the result combination being a combination of a result of the automatically generated finding and a result of the first interpretation finding; and
notifying that is actively notifying predetermined information in response to determination that the result combination is the combination for which the active notification is to be performed.

22. A non-transitory recording medium storing a computer readable program causing a computer to perform:
first acquiring that is acquiring an automatically generated finding obtained by computer processing on medical information;
second acquiring that is acquiring a first interpretation finding created by a user based on the medical information;
determining that is determining whether or not a result combination is a combination for which an active notification is to be performed, the result combination being a combination of a result of the automatically generated finding and a result of the first interpretation finding; and
notifying that is actively notifying predetermined information in response to determination that the result combination is the combination for which the active notification is to be performed.
